(19) United States
(12) Patent Application Publication
(10) Pub. No.: US 2025/0144439 A1
(43) Pub. Date: May 8, 2025

(54) REFLECTOR FOR BIOPHOTON/CELL REACTIVATION

(71) Applicant: Theralogy International GmbH, Waldshut-Tiengen (DE)

(72) Inventor: David Overbeck, Waldshut-Tiengen (DE)

(21) Appl. No.: 18/836,624

(22) PCT Filed: Jan. 30, 2023
(86) PCT No.: PCT/EP2023/052119

§ 371 Date: Aug. 7, 2024

(30) Foreign Application Priority Data
Feb. 8, 2022 (DE) 20 2022 100 700.4

(51) Int. Cl. A61N 5/06

(52) U.S. Cl. CPC A61N 5/0613; A61N 2005/0666; A61N 2005/0667

(57) ABSTRACT

A reflector for biophoton/cell reactivation with a support body containing a receiving surface, with a reflective layer adjoining the receiving surface of the support body and with a filter layer adjoining a side of the reflective layer facing away from the receiving surface, the filter layer being formed selectively by a first filter layer or by a second filter layer, which each allow radiation in a different spectral range to pass through.

The invention relates to a reflector for biophoton/cell reactivation with a support body containing a receiving surface, with a reflective layer adjoining the receiving surface of the support body and with a filter layer adjoining a side of the reflective layer facing away from the receiving surface.

DE 20 2008 011 806 U1 discloses a reflector for biophoton therapy which comprises a support body, a reflective layer and a filter layer. The reflective layer adjoins a receiving surface of the support body. The filter layer is arranged on a side of the reflective layer opposite the receiving surface of the support body. The pot-shaped support body is covered by a glass layer which rests on the filter layer. The disadvantage of the known reflector is that it is relatively bulky and heavy due to the glass cover.

The object of the present invention is to further develop a reflector for biophotons/cell reactivation in such a way that the handling is simplified and the effectiveness is improved.

To solve this problem, the invention in conjunction with the preamble of claim 1 is characterized in that the filter layer is optionally formed by a first filter layer or by a second filter layer, each of which allowing radiation in a different spectral range to pass through.

According to the invention, an upper side of the reflector is formed by a first filter layer or a second filter layer, which differ in that they allow radiation in a different spectral range to pass through. This can advantageously improve the variability of the reflector. Because the reflector essentially consists of the optically effective filter layer and reflection layer (without an additional glass layer), it is relatively light and easy to handle.

According to a preferred embodiment of the invention, the first filter layer is designed in such a way that radiation in a spectral range with a wavelength between 430 nm and 490 nm is allowed to pass through. This allows blue light to pass through.

According to a further development of the invention, the second filter layer is designed such that radiation in a spectral range with a wavelength between 640 nm and 780 nm is allowed to pass through. This allows red light to pass through.

According to a preferred embodiment of the invention, the first filter layer and the second filter layer have the same wall thickness. The reflector thus has the same dimensions regardless of the type of filter layer, so that it is easy to manufacture and easy to use.

According to a further development of the invention, the reflector is designed as a film that has a significantly smaller thickness compared to the first and second filter layers. This advantageously allows the reflector to be constructed relatively flat.

According to a preferred embodiment of the invention, a cover side of the reflector can be connected to an adhesive layer so that the reflector can be detachably attached to a living being with the cover side. The relatively low weight of the reflector counteracts an undesired detachment of the reflector from the skin of the living being.

An embodiment of the invention is explained in more detail below using an embodiment.

The drawings show in:

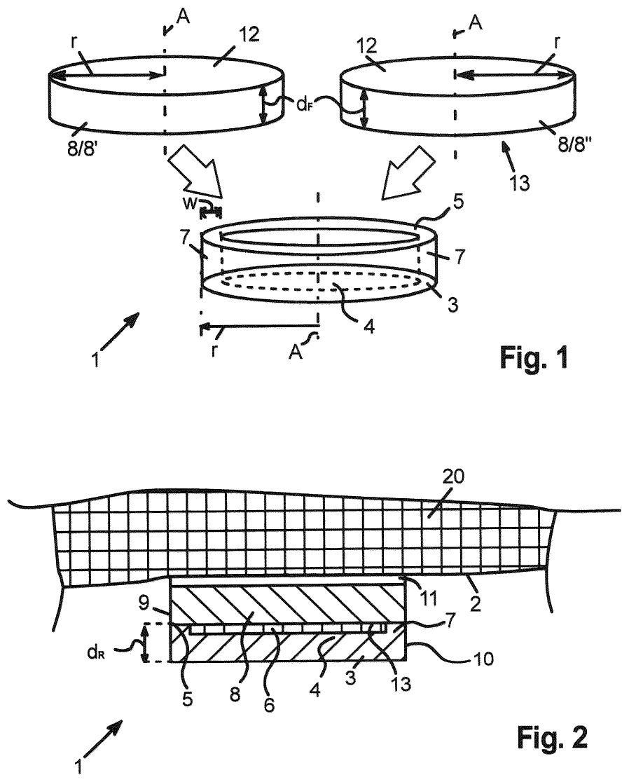

FIG. 1 a perspective view of a reflector without a reflective layer and without an adhesive layer and

FIG. 2 a cross-section through the reflector in a state of use.

A reflector 1 according to the invention is used to attach to the skin 2 of a living being 20: It enables the reactivation of cells as part of cell training (biophoton/cell reactivation). The living beings 20 can be, for example, humans or animals.

The reflector 1 has a support body 3, which is preferably made of a non-transparent material. The support body 3 is plate-shaped with a receiving surface 4 and a narrow rim surface 5. The narrow rim surface 5 is raised in comparison to the receiving surface 4 of the support body 3. The elevation of the narrow rim surface 5 in comparison to the receiving surface 4 is chosen to be so large that a reflection layer 6 is surrounded by a rim 7 of the support body 3 having the narrow surface 5. The reflection layer 6 is preferably adapted to an inner contour of the rim 7 of the support body 3, so that a transverse displacement of the reflection layer 6 perpendicular to an axis of symmetry A of the support body 3 relative to the support body 3 is prevented.

When the reflection layer 6 is inserted, it can run flush with the narrow surface 5 of the rim 7 or, alternatively, can be recessed in comparison to the rim-side narrow surface 5.

The support body 3 is preferably designed to be rotationally symmetrical. In the present exemplary embodiment, the reflector 1 or the support body 3 is cylindrical and/or circular in cross-section.

A filter layer 8 is attached to a side of the reflection layer 6 facing away from the receiving surface 4 of the support body 3.

The filter layer 8 can optionally be designed as a first filter layer 8′ or as a second filter layer 8″, which differ in that the first filter layer 8′ allows radiation in a first spectral range to pass through and the second filter layer 8″ allows radiation in a second spectral range to pass through. The dimensions of the first filter layer 8′ and the second filter layer 8″ are identical.

In the present exemplary embodiment, the first filter layer 8′ is designed such that radiation in a spectral range with a wavelength between 430 nm and 490 nm is allowed to pass through. The second filter layer 8″ is designed such that radiation in a spectral range with a wavelength between 640 nm and 780 nm is allowed to pass through.

The filter layer 8 or the first filter layer 8′ and the second filter layer 8″ are plate-shaped or cylindrical, wherein a wall thickness d_{F} of the filter layer 8, 8′, 8″ can be in a range between 1 mm and 3 mm. A radius r of the filter layer 8, 8′, 8″ is in a range between 1 cm and 3 cm, preferably in a range between 1.8 cm and 2.2 cm, in particular 2 cm.

The wall thickness de of the filter layer 8, 8′, 8″ can be in the range of a wall thickness d_{R} of the rim 7 of the supporting body 3.

In the present embodiment, the radius r of the filter layer 8, 8′, 8″ is equal to a radius r of the support body 3 or the rim 7 thereof. In the assembled state, the filter layer 8, 8′, 8″ rests on the narrow surface 5 of the rim 7, with a peripheral surface 9 of the filter layer 8 running flush with a peripheral surface 10 of the support body 3. The reflector 1 is thus cylindrical.

On a flat side of the filter layer 8, 8′, 8″ facing away from the reflection layer 6, there is provided an adhesive layer 11 that is transparent to light radiation or, in particular, to biophoton radiation, by means of which the support body 3 can be attached to the skin 2 of the living being 20. The adhesive layer 11 is preferably designed as a biocompatible adhesive layer or adhesive film.

According to an alternative embodiment of the invention (not shown), the adhesive layer 11 can be dispensed with. Other fastening means, such as a commercially available adhesive tape that can be used in the medical field, are then required to attach the reflector 1 to the skin 2.

With regard to the adhesive layer 11, it is accepted that the adhesive effect decreases with an increasing number of therapeutic applications of the reflector 1. In order to ensure that the reflector 1 adheres to the skin 2, appropriate adhesive tapes are then required.

According to an embodiment of the invention (not shown), the reflection layer 6 can also be applied to a flat side 13 of the filter layer 8, 8′, 8″ facing the receiving surface 4, for example by vapor deposition, lamination or painting on.

During use, the flat side 12 arranged on the side of the filter layer 8, 8′, 8″ facing away from the reflection layer 6 forms the cover side of the reflector 1 and is the contact surface for resting directly on the skin 2 of the living being. During the therapeutic application, the biophotons pass through the first filter layer 8′ and/or the second filter layer 8″ and are reflected by the reflection layer 6 so that they hit the skin 2 of the living being 20 again via the first filter layer 8′ and/or the second filter layer 8″.

A wall width w of the narrow surface 5 is in a range between 2 mm and 6 mm, preferably in the range of 3 mm to 5 mm. This ensures that the filter layer 8, 8′, 8″ can be attached to the support body 3 in an adhesive manner. The filter layer 8 is preferably attached to the support body 3 by gluing.

1. A reflector for biophoton/cell reactivation with a support body (3) containing a receiving surface (4), with a reflection layer (6) adjoining the receiving surface (4) of the support body (3) and with a filter layer (8, 8′, 8″) adjoining on a side of the reflection layer (6) facing away from the receiving surface (4), wherein the filter layer (8) is selectively formed by a first filter layer (8′) or by a second filter layer (8″), each of which allows radiation in a different spectral range to pass through.

2. The reflector according to claim 1, wherein the first filter layer (8, 8′) is designed such that radiation in a spectral range with a wavelength between 430 nm and 490 nm is allowed to pass through.

3. The reflector according to claim 1, wherein the second filter layer (8, 8″) is designed such that radiation in a spectral range with a wavelength between 640 nm and 780 nm is allowed to pass through.

4. The reflector according to claim 1, wherein the first filter layer (8, 8′) and the second filter layer (8, 8″) have the same wall thickness (dF).

5. The reflector according to claim 1, characterized in that wherein the first filter layer (8, 8′) and the second filter layer (8, 8″) are plate-shaped.

6. The reflector according to claim 1, wherein the wall thickness (d_{F}) of the first filter layer (8′) and the second filter layer (8″) is in the range from 2 mm to 10 mm.

7. The reflector according to claim 1, characterized in that wherein the reflection layer (6) is designed as a white-colored paper disc or white-colored plastic disc.

8. The reflector according to claim 1, wherein the reflection layer (6) is vapor-deposited, laminated or painted onto a flat side (13) of the first filter layer (8′) and the second filter layer (8″).

9. The reflector according to claim 1, characterized in that wherein a flat side (12) of the filter layer (8, 8′, 8″) arranged on the side facing away from the reflection layer (6) is connected to a light-transparent or biophoton-transparent adhesive layer (11) for fastening the reflector (1) to a skin (2) of a living being (20).

10. The reflector according to claim 9, wherein the adhesive layer (11) is a biocompatible adhesive film.

11. The reflector according to claim 1, wherein the first filter layer (8, 8′) and the second filter layer (8, 8″) are cylindrical.

12. The reflector according to claim 1, wherein the wall thickness (d_{F}) of the first filter layer (8′) and the second filter layer (8″) is in the range from 1 mm to 3 mm.

13. The reflector according to claim 1, wherein the reflection layer (6) is vapor-deposited, laminated or painted onto a flat side (13) of the second filter layer (8″).
